(19) United States
(12) Patent Application Publication
(10) Pub. No.: US 2011/0290245 A1
(43) Pub. Date: Dec. 1, 2011

(54) Internally Braced Radial Balloon Dilator

(76) Inventors: Brian J. Cuevas, Cumming, GA (US); Michael Sleva, Atlanta, GA (US); Joseph A. Cesa, Cumming, GA (US)

(21) Appl. No.: 12/788,409

(22) Filed: May 27, 2010

(51) Int. Cl. A61M 16/04; A61M 29/02

(52) U.S. Cl. 128/200.26

(57) ABSTRACT

There is provided a device for performing a balloon dilation tracheostomy. The balloon has internal braces or “stays” extending from the center to the inside surface of the balloon, though other arrangements are possible. The braces control the ultimate expansion and final shape of the balloon when it is fully inflated, inhibiting the ability of parts of the balloon to herniate. The balloon should also expand more uniformly, allowing the practitioner to more confidently expand the tracheal stoma site and ensuring a successful dilation and tube placement.

Ventilators or respirators are used for mechanical ventilation of the lungs of a patient in a medical setting. The ventilator unit is connected to a hose set; the ventilation tubing or tubing circuit, delivering the ventilation gas to the patient. At the patient end, the ventilation tubing is typically connected to a tracheal ventilation catheter or tube, granting direct and secure access to the lower airways of a patient. Tracheal catheters are equipped with an inflated sealing balloon element, or “cuff”, creating a seal between the tracheal wall and tracheal ventilation tube shaft, permitting positive pressure ventilation of the lungs.

One type of tracheal catheter, an endotracheal tube (ET tube), inserted through the mouth, is generally used for a number of days before a decision is made to switch a patient to a tracheostomy tube, inserted directly into the trachea through a stoma in the tracheal wall. Endotracheal tubes have been linked in some studies to an increased rate of ventilator acquired pneumonia (VAP) and so tracheostomy operations are becoming increasingly common and are being performed earlier in the patient's hospital stay in order to reduce the occurrence of VAP.

A tracheostomy procedure involves making a small horizontal incision in the skin of the neck to grant access to the trachea. After the skin incision, a hemostat or other implement may be used to separate the subcutaneous tissues to gain access to the trachea, and digital palpation is used to locate the tracheal rings. A sheathed needle is used to puncture the tracheal wall, usually between the second and third tracheal rings. The needle is removed with the sheath remaining, a flexible guide wire (also called a J-wire) is inserted in the place of the needle and the sheath is removed. A common method of trachoestomy practiced involves the use of a rigid plastic dilator, such as the Cook Medical Inc. Blue Rhino® dilator (see also U.S. Pat. No. 6,637,435). The dilator is placed over the guide wire and advanced into the trachea through the tracheal wall to perform the dilation. Cook Medical recommends a slight over-dilation of the tracheal wall in order to make the placement of the tracheostomy tube easier. After dilation, the dilator is removed and the tracheostomy tube (with cannula removed) is introduced over the guide catheter using an insertion tool that fits just inside the tracheostomy tube. Once the tracheostomy tube is at the proper depth, the insertion tool and guide wire are removed through the tracheostomy tube, the inner cannula inserted into the tracheostomy tube and the tube is connected to the ventilator.

Dilation using an inflatable balloon is an alternative method. U.S. Pat. No. 5,653,230, for example, teaches balloon dilation. In this procedure a collapsed balloon is inserted into the incision in the tracheal wall and slowly inflated, forcing the tracheal rings apart as it inflates. While a number of medical practitioners prefer this method, this procedure has been found to have a number of challenges. One problem is that the balloon may be punctured by the tracheal rings and rendered useless. Another problem is that the balloon may inflate only partially or the portion within the trachea may inflate while leaving the portion of the balloon directly in the stoma site un-inflated. If this occurs, since the practitioner is monitoring the pressure in the balloon as a guide to the dilation, the pressure may be correct but the balloon may be inflated in an area unseen by the practitioner. Additional pressure may be applied, resulting in the rupture of the balloon in the trachea without dilation of the tracheal wall. Short of rupture, should the balloon herniate within the trachea unbeknownst to the practitioner, undue pressure can be placed on the tracheal wall by the expanded portion of the balloon. This can result in compression of the tissue in the wall of the trachea, possibly resulting in stenosis.

Attempts have been made to address the first problem, as described for example, by U.S. patent application Ser. No. 11/044,773. This application teaches a balloon dilator having a reinforced balloon that resists puncture by using a braided pattern of reinforcement wires, preferably stainless steel, wound around the balloon. The balloon, however, can still conform to the shape of internal passageways, allowing the possibility that the balloon may expand, sight unseen, in the trachea. Still another example of a reinforced balloon dilator may be found in U.S. Pat. No. 5,772,681.

As can be understood from the above description, while there are a number of procedures, the current state of the art for tracheostomy involves numerous steps and the insertion and removal of a number of components before the successful completion of the procedure. For most of this time, the patient is disconnected from the ventilator and is therefore, not breathing. This is clearly a significant event, especially for a patient who is, most likely, not in optimal physical condition. In the balloon dilation procedure, should the balloon expand within the trachea instead of in the tracheal stoma, the time of the procedure can be extended greatly, putting the patient at further risk.

There remains a need for a device that can more quickly and reliably allow for the successful dilation of the tracheal wall and placement of a tracheostomy tube.

### SUMMARY OF THE INVENTION

There is provided a device for performing a balloon dilation tracheostomy. The device has a balloon that dilates a stoma where the balloon includes internal braces that limit the expansion of the balloon and so impede herniation or over-inflation of a part of the balloon without over-inflation of other expandable parts of the balloon. The balloon inflates in a more controlled manner, thus allowing the practitioner to more confidently dilate the tracheal stoma site and place the tracheostomy tube.

### BRIEF DESCRIPTION OF THE DRAWINGS

FIG. 1 is a drawing of a percutaneous dilation device as disclosed herein.

FIG. 2 is a drawing of a punch dilator with a hollow needle being used to make the initial puncture of the tracheal wall.

FIG. 3 is a drawing showing a guidewire inserted in place of the needle in the punch dilator, through the sheath and into the trachea.

FIG. 4 is a drawing of a percutaneous dilation device that is positioned over the guidewire and advanced along the guidewire until the dilation balloon of the device is positioned in the stoma site.

FIG. 5 is a drawing of the device of FIG. 4 with the dilation balloon inflated to expand the stoma.

FIG. 6 is a drawing of a percutaneous dilation device that has its dilation balloon deflated and has been moved distally into the trachea to position the tracheal tube in the stoma.

FIG. 7 is a drawing of a tracheal tube in its final position with the cuff expanded.

FIG. 8 is a drawing of a dilation balloon and braces about a shaft shown in cross section.

FIGS. 9 through 12 are drawings of other dilation balloons about supportive shafts with examples of braces.

### DETAILED DESCRIPTION OF THE INVENTION

Tracheostomy is a lifesaving procedure to allow a patient to be ventilated directly through the trachea. Tracheostomy is also believed by many to prevent or retard the onset of ventilator acquired pneumonia (VAP). This lifesaving procedure, unfortunately, is relatively time consuming and current technology requires a large number of steps and pieces of equipment that must remain sterile and functioning properly in order to arrive at a successful conclusion.

Tracheostomy procedures can be improved using the device as described in the Summary above. The device includes a balloon that is intended to dilate a stoma where the balloon is limited in its ultimate expansion about a supporting shaft by braces that attach to the inside surface of the balloon. The braces control the ultimate expansion and final shape of the balloon when it is fully inflated, inhibiting the ability of parts of the balloon to herniate. This may also result in a more uniform expansion. Controlling the ultimate expansion of the balloon reduces the possibility that a portion of the balloon may herniate and cause the serious problems discussed in the Background above.

Examples of suitable braces include stays, strands, tie-backs, walls, and baffles that extend from the inside surface of the balloon to the shaft enclosed by the balloon or to another inside surface of the balloon or both.

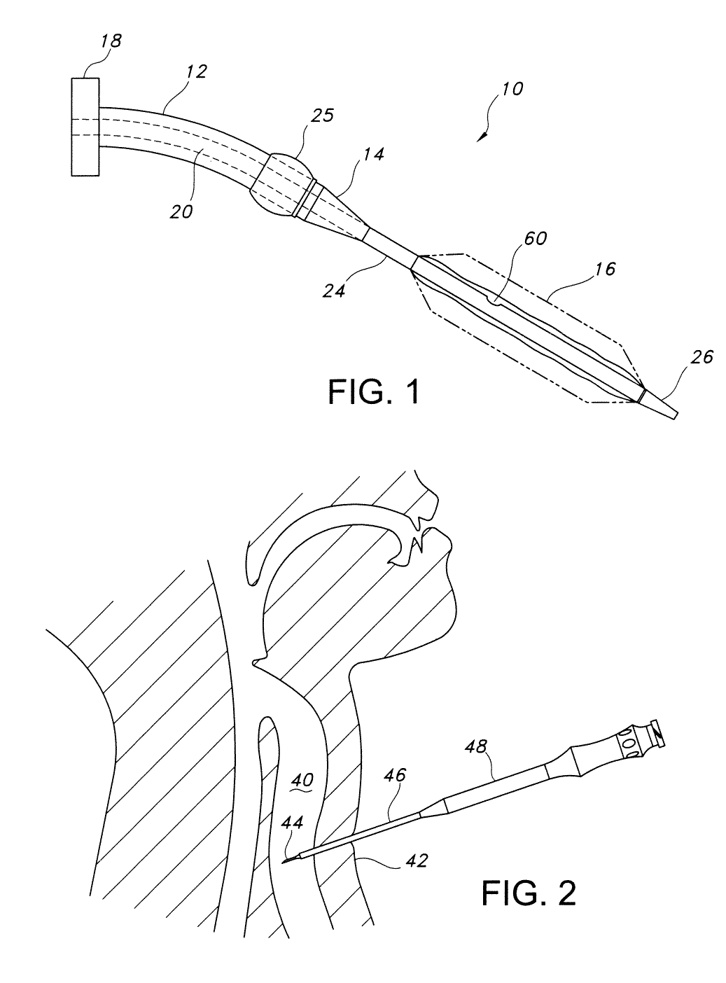

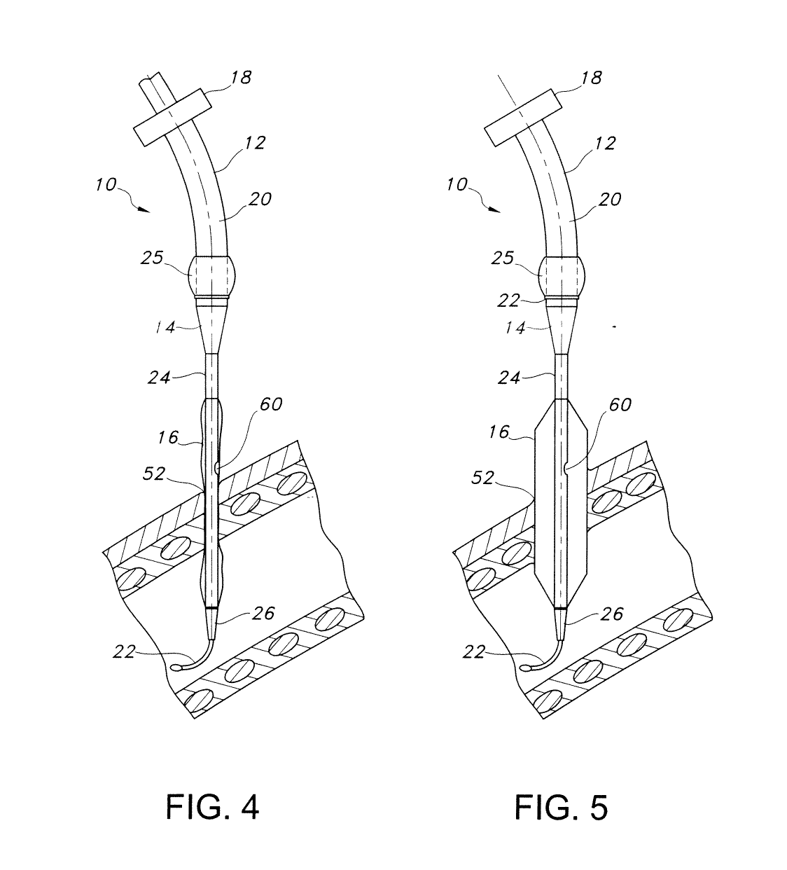

Turning to FIG. 1, one embodiment of the percutaneous dilation device 10 has a hollow trachestomy tube 12 with a distal end 14 that is preferably tapered to allow for easy insertion into a tracheal stoma. Extending through and beyond the tube 12 is shaft 24 and the dilation balloon 16. The balloon 16 has a distal end and a proximal end that define a maximum length of the balloon along the shaft 24. The shaft 24 extends through the tube 12 so that the shaft 24 and balloon 16 may be removed through the tube 12 once the stoma has been successfully dilated. The shaft 24 also has a hollow bore or cannula 20 for the insertion and removal of a guidewire 22 (shown in FIGS. 4, 5 and 6). The tube 12 may also have a flange 18 to limit the insertion of the tube 12 into the trachea and also for securing the tube 12 to the patient. The trachestomy tube 12 may also have a balloon, hereinafter called a cuff 25 to avoid confusion with the dilation balloon 16. The cuff 25 is attached directly to the tracheostomy tube 12 and is used to block the flow of air around the tube 12 when it is in service in the trachea.

The diameter of the balloon 16 when inflated should be selected based on the size of the tracheal tube 12 being inserted. For example, for use with a tracheal tube 12 having an outside diameter of 12.0 mm, the balloon 16 should have a diameter of 12.0 mm when inflated, or slightly more, for example 0.5 to 1.0 mm more. This close sizing or slight over-sizing of the balloon diameter as compared to the tracheal tube diameter helps ensure that the opening formed by the balloon 16 will be large enough to prevent damage to the cuff 25 during insertion of the tracheal tube 12 into the stoma.

It is desirable that the tracheal tube 12, the shaft 24, and the dilation balloon 16 be adapted for advancement along the guidewire 22 together in a unitary fashion that is without any longitudinal movement relative to one another during advancement of the device 10. Movement in this fashion reduces the number of manipulative steps necessary to introduce the tracheal tube 12 into the stoma, and thereby makes the placement of the tube 12 faster and easier to perform.

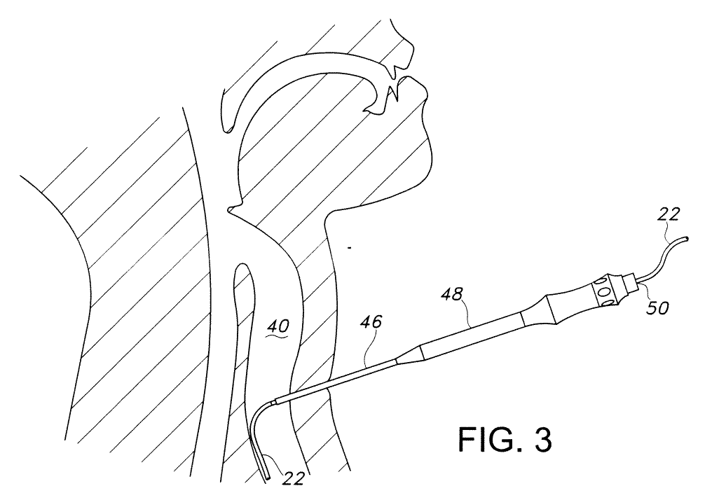

The method of performing a tracheostomy according to the current disclosure will now be described in greater detail than is mentioned above. The cricothyroid membrane of the tracheal wall 42 between the thyroid and cricoid cartilages is first palpated, and a slight incision is made with a scalpel through the skin and the membrane. As shown in FIG. 2, a punch dilator 48 with a hollow needle 44 surrounded by at least a sheath 46 may be used to make the initial puncture of the tracheal wall 42. Once it has been determined that the needle 44 has indeed entered the trachea 40, the needle 44 is withdrawn from the trachea 40 and from the sheath 46, leaving the sheath 46 in place in the stoma. As shown in FIG. 3, the guidewire 22 or “J-wire” may then be inserted through the sheath 46 into the trachea 40. The guidewire 22 is introduced to the trachea 40 through the central cannula or lumen of the sheath 46 via the proximal opening 50 of the sheath 46. The punch dilator 48 may optionally be advanced further into the stoma to expand the stoma slightly prior to insertion of the percutaneous dilation device 10. The entire dilator 48 is then removed, leaving only the guidewire 22 in place in the stoma. Other methods of creating the initial puncture of the tracheal wall may employ needles and syringes in lieu of the punch dilator. One particularly suitable device for making the initial puncture is described in U.S. patent application Ser. No. 12/475,754.

As shown in FIG. 4, the next step in the method is to position the dilation balloon 16 over the guidewire 22. Such positioning is carried out by placing the tip 26 of the device 10 over the guidewire 22 so that the guidewire 22 is received in the cannula 20 in the device 10. The dilation balloon 16, carried by the device 10, is then advanced along the guidewire 22 toward the patient until the dilation balloon 16 of the device 10 is positioned in the stoma site 52.

An inflation source for the balloon 16 (not shown) is then activated to supply fluid into the dilation balloon 16 through a balloon inflation cannula terminating in port 60 within the balloon 16. The balloon 16 expands generally perpendicularly to the shaft 24 to enlarge the stoma 52 in the tracheal wall 42 (FIG. 5). Once the stoma 52 is expanded, the balloon 16 may be deflated by withdrawing fluid in the opposite operation as it was supplied. The device 10 as described above generally includes a tracheostomy tube 12. The dilation balloon 16 may, however, be used separately from the tube 12 if desired. If the balloon 16 is used separately, it will be necessary at this point to remove the balloon 16 and shaft 24 from the stoma 52 and withdraw it from the guidewire 22, leaving the guidewire 22 in place in the stoma, and to slide the tube 12 over the guidewire 22 and into the stoma 52 to the point where the flange 18 of the tube 12 is resting against the skin of the throat of the patient. The guidewire 22 may be withdrawn through the tube 12, the cuff 25 inflated and the tube 12 placed in service.

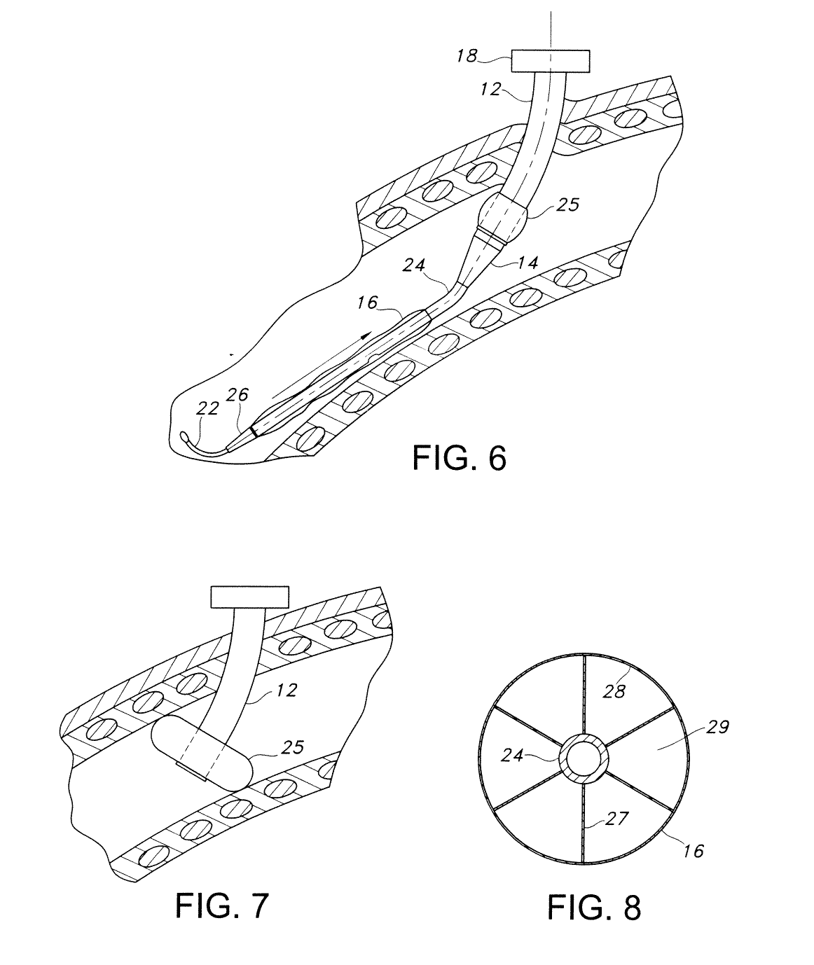

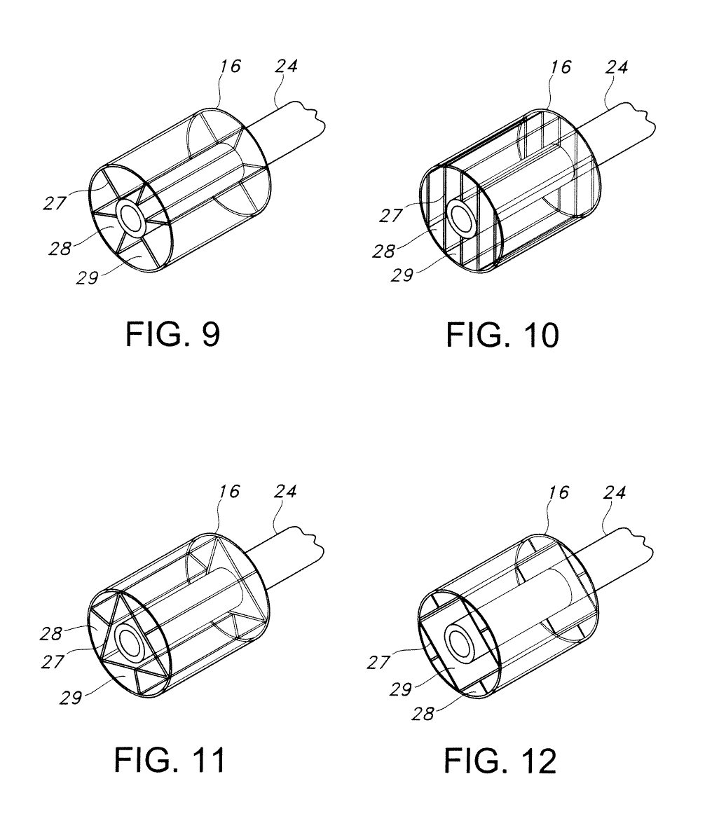

If the balloon 16 is used with the entire device 10, the device 10 is manually advanced into the trachea 40 (FIG. 6) to the point where the flange 18 of the tube 12 is resting against the skin of the throat of the patient. The device 10 is manipulated so that the tracheal tube 12 detaches from shaft 24 and the balance of the device 10 (i.e. without the tracheal tube 12) and the guidewire 22 may then be withdrawn through the tracheal tube 12.

Whichever method of insertion of the tube 12 is used, once the trach tube 12 is in position in the trachea, the cuff 25 on the trach tube 12 may be inflated by conventional means and the trach tube 12 placed in service (FIG. 7).

An example of a dilation balloon 16 suitable for use in the invention is shown in cross section in its fully inflated state in FIG. 8. This embodiment shows six braces 27 that tie the inner surface 28 of the balloon 16 to the central shaft 24 by spanning between them. Alternatively, the braces may span from a first location on the inner surface 28 of the balloon 16 to a second location on the inner surface 28 of the balloon 16, as visible in FIGS. 11 and 12. A combination of braces, with some spanning from the central shaft 24 to the inner surface 28 of the balloon 16 and some spanning from a first location on the inner surface 28 of the balloon 16 to a second location on the inner surface 28 of the balloon 16, as shown in FIG. 10, may also be used. Desirably, the braces may span from the distal end of the balloon to the proximal end of the balloon.

There may be as few as three braces 27 and as many as desired, depending on the application. It is anticipated that more than nine braces would be unnecessary in most applications, however. The braces 27 need not be solid and may have gaps, holes or perforations along their length, which may allow for more efficient inflation of the balloon 16.

The braces 27 need not be continuous along the length of the balloon 16 and may instead be strands, threads or fibers connected between the shaft 24 and the balloon inner surface 28. When the braces are strands, they need not be aligned or spaced uniformly and their number may of course be much higher than nine.

The balloon 16 desirably has a generally cylindrical shape when fully inflated. The balloon 16 may also have a curved, banana-like shape produced by the adjustment of the length of the braces 27. A more curved shape may make insertion into the trachea easier. Such shaping can result from intentional variations in the arrangement of the braces, in the spanning dimensions of the braces, or both within the balloon.

The balloon 16 may optionally also have a braided reinforcement system as described in U.S. patent application Ser. No. 11/044,773. This system uses wires running circumferentially around the balloon 16 to protect the balloon from punctures from splintered tracheal rings or other sources. Various patterns of wire braiding may be used, such as helical braiding, and the wires may be made from metals, alloys (e.g. stainless steel) and non-metallic compositions.

The balloon 16 and the braces 27 may be made at the same time, for example by extrusion of a molten polymer through a die. FIG. 8 shows an example of a cross-sectional profile of a balloon with symmetrically arranged and spaced braces that span from the interior surface of the balloon towards the center of the balloon. The distal and proximal ends of the balloon 16 may be closed by the application of heat or through the use of an adhesive. These ends can be formed at the same time the balloon is attached to the shaft 24 or the ends can be formed prior to such attachment. The section of shaft 24 having the inflation port 60 may be inserted through the balloon 16 and the balloon 16 fixed in place, again with heat, adhesive or other known method. Alternatively, the braces 27 may be made separately from balloon 16 and these parts joined together by adhesives or heat. When there are braces that mostly traverse between the ends of the balloon, they form compartments 29. When compartments are formed, it is important that they be in fluid communication with one another or with a port 60 so that the balloon 16 may be inflated and deflated. The fluid communication can be direct, for example when each compartment communicates with its own port 60, or indirect, such as when one compartment is in direct fluid communication with another compartment that has fluid communication with a port 60.

As mentioned above, the trach tube 12 has a cuff 25 around its circumference on a lower (distal) portion of the tube that serves to block the normal air flow in the trachea so that (assisted) breathing takes place through the tracheostomy tube using a ventilator. The cuff 25 is desirably made from a soft, pliable polymer such as polyurethane (PU), polyethylene teraphithalate (PETP), low-density polyethylene (LDPE), polyvinyl chloride (PVC), or elastomeric-based polyolefins. It should be very thin; on the order of 25 microns or less, e.g. 20 microns, 15 microns, 10 microns or even as low as 5 microns in thickness. The cuff 25 should also desirably be a low pressure cuff 25 operating at about 30 mmH₂O or less, such as 25 mmH₂O, 20 mmH₂O, 15 mmH₂O or less. Such a cuff 25 is described in U.S. Pat. No. 6,802,317 which describes a cuff 25 for obturating a patient's trachea as hermetically as possible, comprising a cuff 25 which blocks the trachea below a patient's glottis, an air tube, the cuff 25 being attached to the air tube and being sized to be larger than a tracheal diameter when in a fully inflated state and being made of a soft, flexible foil material that forms at least one draped fold in the cuff 25 when inflated in the patient's trachea, wherein the foil has a wall thickness below or equal to 0.01 mm and the at least one draped fold has a loop found at a dead end of the at least one draped fold, that loop having a small diameter which inhibits a free flow of secretions through the loop of the at least one draped fold. Another description of such a cuff 25 is in U.S. Pat. No. 6,526,977 which teaches a dilator for obturating a patient's trachea as hermetically as possible, comprising a cuff 25 which blocks the trachea below a patient's glottis, a trach tube, the cuff 25 being attached to the trach tube and being sized to be larger than a tracheal diameter when in a fully inflated state and being made of a sufficiently soft, flexible foil material that forms at least one draped fold in the cuff 25 when fully inflated in the patient's trachea, wherein the at least one draped fold formed has a capillary size which arrests free flow of secretions across the cuff 25 by virtue of capillary forces formed within the fold to prevent aspiration of the secretions and subsequent infections related to secretion aspiration.

Alternatively, the cuff 25 may be of a shape as described in U.S. patent application 60/994,664, now Ser. No. 12/206,517 or U.S. Pat. No. 6,612,305. In the '305 patent, the cuff 25 expands in the trachea not only around the tube 12, as do the current models, but also cranially to it and to the stoma, sealing the stoma. Sealing of the stoma in the '305 device is achieved by the fact that the proximal point of attachment and the distal point of attachment of the inflatable cuff 25 on the tube 12 are not contiguous or, in other words, are at an angle (α) other than 180 degrees, relative to conventional devices.

In the '644 application, the cuff 25 has a distal portion substantially centered about and attached to the distal end portion of the tube 7. The cuff 25 also has a proximal portion attached to the bend region of the tube and positioned substantially off-center about the bend region below the proximal plane of the device. Upon inflation, this configuration provides for expansion of the cuff 25 around the distal end portion of the tube and the proximal end portion of the tube below the proximal plane of the device to seal the trachea below the tracheal stoma 66 and avoid sealing the trachea above the tracheal stoma. Desirably, this configuration of the cuff 25 will allow secretions to exit the stoma.

The tracheostomy tube 12 cuff 25 may have walls that are non-uniform in thickness. For example, the device may have a first portion of the cuff 25 in which the walls have a thickness of about 20 to 30 microns and a second portion of the cuff 25 in which the walls have a thickness of about 5 to about 15 microns. Desirably, the first portion of the cuff 25 is the portion of the cuff 25 contacting the upper portion of a cross-sectional region of the tracheal lumen and the second portion of the second cuff 25 is the portion of the cuff 25 contacting the lower portion of the same cross-sectional region of the tracheal lumen.

The inflatable cuff 25 may include a distal end, a distal attachment zone, a proximal end, a proximal attachment zone, an upper region and a lower region, wherein the upper region has a thickness of from about 15 to about 30 micrometers and the lower region has a thickness of from about 5 to about 15 micrometers.

The cuff 25 may, as mentioned briefly above, be formed from thermoplastic polyurethane polymers, thermoplastic polyolefin elastomers, thermoplastic polyolefin block copolymers, SBS di-block elastomers, SEBS tri-block elastomers, polyvinyl chloride, polyethylene terephthalate and blends and mixtures thereof as well as any other suitable polymer.

Another optional trauma reducing feature is a lubricious coating that may be added to the tip 26, dilator balloon 16, trach tube 12 and cuff 25. The coating may be activated by exposure to water before the device 10 is slipped over the guidewire 22. The coating may be for example, a poly(N-vinyl) lactam such as those available from Hydromer Inc., 35 Industrial Parkway, Branchburg, N.J. and as described in U.S. Pat. Nos. 5,156,601, 5,258,421, 5,420,197 and 6,054,504. The items may be dipped in water just before the J-wire is inserted and may be coated on the inside and/or outside. An inside coating allows the J-wire to slip through the interior of the dilator quite easily and the exterior coating avoids trauma to the skin or trachea.

The device 10 must further be biocompatible, free of di(2-ethylhexyl) phthalate (DEHP) and preferably free of animal derived products.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A balloon dilator for performing a tracheostomy comprising a balloon having internal braces, wherein at least one of the braces connects a central shaft to an inner surface of said balloon.

2. The dilator of claim 1 wherein there are from three to nine braces.

3. The dilator of claim 1 wherein the braces are perforated.

4. The dilator of claim 1 wherein the braces are stays, strands, threads or fibers.

5. The dilator of claim 1 wherein at least one of said braces span from a distal end of the balloon to a proximal end of the balloon.

6. A balloon dilator for performing a tracheostomy comprising a balloon having internal braces, wherein said braces span from a first location on an inner surface of the balloon to a second location on the surface of the balloon.

7. A device for performing a percutaneous dilation and placement of a tracheostomy tube comprising a hollow trachestomy tube with a distal end, a shaft upon which is located a dilation balloon with internal braces, the shaft extending through the tube, the shaft having a hollow bore or cannula for the insertion and removal of a guidewire.

8. The device of claim 7 wherein the tube has a flange to limit the insertion of the tube into the trachea and also for securing the tube to a patient, the tube also having an inflatable cuff to block the flow of air around the tube after inflation.
